(19) United States
(12) Patent Application Publication
(10) Pub. No.: US 2022/0120743 A1
(43) Pub. Date: Apr. 21, 2022

(54) FLOW CONTROL LINES FOR LATERAL FLOW ASSAYS

(71) Applicant: Detect, Inc., Guilford, CT (US)

(72) Inventors: Amy Cornell, Killingworth, CT (US); Todd Roswech, Ivoryton, CT (US); John H. Leamon, Stonington, CT (US)

(73) Assignee: Detect, Inc., Guilford, CT (US)

(21) Appl. No.: 17/502,999

(22) Filed: Oct. 15, 2021

Related U.S. Application Data
(60) Provisional application No. 63/092,837, filed on Oct. 16, 2020.

(51) Int. Cl. G01N 33/543; B01L 3/00

(52) U.S. Cl. CPC G01N 33/54388; B01L 3/5023; B01L 2200/16; B01L 2300/069; B01L 2300/0825

(57) ABSTRACT

Described herein in an embodiment is a lateral flow assay strip for detecting the presence of one or more target nucleic acid sequences (e.g., one or more nucleic acid sequences of a pathogen, such as a coronavirus or an influenza virus). The lateral flow assay strip may comprise a first test line configured to detect a first target nucleic acid sequence and a flow control line configured to detect a liquid. In some cases, the first test line comprises a biological capture reagent (e.g., an immobilized antibody). In some cases, the flow control line comprises a non-biological fluid indicator (i.e., a non-antibody reagent). The non-biological fluid indicator may, for example, comprise a pH-sensitive material, a moisture-sensitive material, and/or a chemically-sensitive material. The flow control line may provide a visual indication of whether a fluidic sample was successfully transported from a first end of the lateral flow assay strip to the first test line.

### RELATED APPLICATION

This application claims the benefit under 35 U.S.C. § 119(e) of U.S. Provisional Patent Application No. 63/092,837, filed Oct. 16, 2020, and entitled “Lateral Flow Assay,” which is incorporated by reference herein in its entirety.

### FIELD

The disclosure generally relates to lateral flow assay strips comprising flow control lines.

### BACKGROUND

The ability to rapidly diagnose infectious diseases is critical to protecting human health. As one example, the lack of adequate testing for the highly contagious novel coronavirus disease 2019 (COVID-19) has contributed to a global pandemic that has killed millions of people. The existence of a rapid, accurate COVID-19 diagnostic test could allow infected individuals to be quickly identified and isolated, which could facilitate containment of the disease and treatment of infected individuals.

### SUMMARY

Provided herein are lateral flow assay strips comprising flow control lines.

Some aspects are directed to a lateral flow assay strip. In some embodiments, the lateral flow assay strip comprises a substrate having a first end and a second end. In certain embodiments, the substrate comprises a first test line configured to detect a first target nucleic acid sequence. In certain embodiments, the substrate comprises a flow control line comprising a first non-biological fluid indicator. In some cases, the flow control line is positioned between the first test line and the second end of the substrate. In some cases, the substrate is configured to transport a fluidic sample from the first end to the second end of the substrate.

Some aspects are directed to a diagnostic system. In some embodiments, the diagnostic system comprises one or more nucleic acid amplification reagents. In certain embodiments, the one or more nucleic acid amplification reagents comprise a first primer directed to the first target nucleic acid sequence. In some embodiments, the diagnostic system comprises a lateral flow assay strip.

Some aspects are directed to a diagnostic method. In some embodiments, the diagnostic method comprises performing an isothermal nucleic acid amplification reaction configured to amplify a first target nucleic acid sequence. In some embodiments, the diagnostic method comprises exposing a fluidic product of the isothermal nucleic acid amplification reaction to a lateral flow assay strip having a first end and a second end. In some embodiments, the lateral flow assay strip comprises a first test line configured to detect the first target nucleic acid sequence. In some embodiments, the lateral flow assay strip comprises a flow control line comprising a first non-biological fluid indicator. In certain embodiments, the flow control line is positioned between the first test line and the second end of the substrate.

Some aspects are directed to a method of preparing a lateral flow assay strip. In some embodiments, the method comprises contacting a substrate having a first end and a second end with one or more capture reagents to form a first test line at a first location on the absorbent substrate. In some embodiments, the method further comprises contacting the substrate with a non-biological fluid indicator to form a flow control line at a second location on the absorbent substrate, wherein the second location is positioned between the first test line and the second end of the substrate.

The foregoing and other aspects, embodiments, and features of the present technology can be more fully understood from the following description in conjunction with the accompanying drawings.

### BRIEF DESCRIPTION OF THE DRAWINGS

A skilled artisan will understand that the accompanying drawings are for illustration purposes only. It is to be understood that in some instances various aspects of the present technology may be shown exaggerated or enlarged to facilitate an understanding of the invention. In the drawings, like reference characters generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the present teachings in any way.

FIG. 1A shows, according to some embodiments, a schematic illustration of a lateral flow assay strip comprising a first test line and a flow control line;

FIG. 1B shows, according to some embodiments, a schematic illustration of a lateral flow assay strip comprising a first test line, a nucleic acid control line, and a flow control line;

FIG. 1C shows, according to some embodiments, a schematic illustration of a lateral flow assay strip comprising a first test line, a second test line, a nucleic acid control line, and a flow control line;

FIG. 2 shows a schematic illustration of an exemplary “chimney-type” diagnostic apparatus, according to some embodiments;

FIG. 3 shows, according to some embodiments, a schematic illustration of an exemplary diagnostic system; and

FIGS. 4A-D show images of an exemplary lateral flow assay strip comprising a first test line, a nucleic acid control line, and a non-biological flow control line under no sample flow conditions (FIG. 4A) and after 5 minutes of sample flow (FIG. 4B), 10 minutes of sample flow (FIG. 4C), and 15 minutes of sample flow (FIG. 4D).

### DETAILED DESCRIPTION

Described herein in an embodiment is a lateral flow assay strip for detecting the presence of one or more target nucleic acid sequences (e.g., one or more nucleic acid sequences of a pathogen, such as a coronavirus or an influenza virus). The lateral flow assay strip may comprise a first test line configured to detect a first target nucleic acid sequence and a flow control line configured to detect a liquid. In some cases, the first test line comprises a biological capture reagent (e.g., an immobilized antibody). In some cases, the flow control line comprises a non-biological fluid indicator (i.e., a non-antibody reagent) that exhibits a visible change (e.g., a change from a first color to a second color, an opaque band becoming more or less visible) upon contact with a fluidic sample. The non-biological fluid indicator may, for example, comprise a pH-sensitive material, a moisture-sensitive material, and/or a chemically-sensitive material. In some cases, flow control lines comprising a non-biological fluid indicator provide an effective, low-cost approach for determining whether a fluidic sample was successfully transported through a lateral flow assay strip (i.e., determining whether a test was valid).

In some embodiments, the lateral flow assay strip has a first end and a second end, and the lateral flow assay strip is configured to transport a fluidic sample (e.g., a sample comprising one or more fluids) from the first end to the second end. The flow control line may be positioned between the first test line and the second end of the lateral flow assay strip and may exhibit a visible change (e.g., a change from a first color to a second color, an opaque band becoming more or less visible) upon contact with a liquid (e.g., a fluidic sample). The presence or absence of a visible change of the flow control line may therefore indicate whether a fluidic sample introduced at the first end of the lateral flow assay strip was successfully transported beyond the first test line to the flow control line. The presence of a visible change of the flow control line may indicate that the fluidic sample successfully reached the flow control line and therefore the first test line, while the absence of a visible change of the flow control line may indicate that the fluidic sample did not reach the flow control line. The absence of a visible change of the flow control line may indicate that a diagnostic test employing the lateral flow assay strip was invalid and may need to be repeated.

Some lateral flow assay strips known in the art comprise a flow control line comprising one or more biological capture reagents (e.g., immobilized antibodies). For example, some known lateral flow assay strips comprise a first test line comprising one or more biological capture reagents and a flow control line comprising one or more biological capture reagents. However, flow control lines comprising biological capture reagents may be negatively impacted by competition with targets binding to the upstream first test line (and, in some cases, one or more additional upstream test and/or control lines also comprising biological capture reagents). That is, the first test line (and, in some cases, one or more additional upstream test lines and/or control lines) may limit the strength of the signal available at the flow control line. In addition, flow control lines comprising biological capture reagents may be relatively expensive.

Some aspects of the present disclosure are directed to a lateral flow assay strip comprising a flow control line that employs a different detection mechanism than the first test line (and, in some cases, one or more additional upstream test lines and/or control lines). In some embodiments, a lateral flow assay strip comprises one or more upstream test and/or control lines comprising one or more biological capture reagents. In some embodiments, the lateral flow assay strip comprises a flow control line comprising one or more non-biological fluid indicators. In some embodiments, the one or more non-biological fluid indicators comprise a pH-sensitive material, a moisture-sensitive material (i.e., a hydrochromic material), and/or a chemically sensitive material. In some cases, a non-biological fluid indicator advantageously provides a flow control line that reliably produces a visible change upon contact with a liquid. For example, a flow control line comprising a non-biological fluid indicator may not be negatively impacted by competition with one or more upstream test and/or control lines. As a result, a flow control lines comprising a non-biological fluid indicator may consistently produce visible, high-contrast bands when contacted by a liquid. In some cases, a flow control line comprising a non-biological fluid indicator may advantageously be less expensive than a flow control line comprising a biological capture reagent.

The present disclosure further provides diagnostic systems comprising a lateral flow assay strip described herein and methods of using the diagnostic systems to detect one or more target nucleic acid sequences. In some embodiments, the diagnostic systems comprise a diagnostic apparatus configured to be used with a lateral flow assay strip. Non-limiting examples of a suitable diagnostic apparatus include a “chimney” type diagnostic apparatus, a cartridge diagnostic apparatus, a blister pack diagnostic apparatus, and an integrated swab diagnostic apparatus. In some embodiments, the diagnostic systems comprise one or more components or devices usable by or in the diagnostic apparatus (e.g., a sample-collecting component such as a swab, a reaction tube or other reaction vessel, a reaction tube cap such as a caged cap, a heater). In some embodiments, the diagnostic systems comprise one or more reagents (e.g., lysis reagents, nucleic acid amplification reagents, CRISPR/Cas detection reagents, buffers). In some embodiments, the diagnostic methods include procedures for using any one or any combination of the diagnostic apparatuses, components or devices, or reagents mentioned above and/or described herein. In some embodiments, a test kit may comprise any combination of one or more of the diagnostic apparatuses, components or devices, or reagents mentioned above and/or described herein, and may include instructions for a user to use the various parts of the test kit and/or to read a diagnostic result manually or with the aid of an application installed on an electronic device.

Methods of using a diagnostic system comprising a lateral flow assay strip described herein may be performed by a trained medical professional in a point-of-care setting (e.g., a hospital, a doctor's office), a lay person (e.g., a person not trained in medical or laboratory techniques) in a non-clinical setting (e.g., a home, school, office, library, store), and/or a subject of a diagnostic test (i.e., a person may self-administer a diagnostic test). Given the expectation that the diagnostic systems described herein may be used in a variety of settings, including both point-of-care and non-clinical settings, it may be particularly important to provide a robust flow control line that can consistently indicate whether a method of using a diagnostic system was properly performed.

### Lateral Flow Assay Strip

Some embodiments are directed to a lateral flow assay strip (e.g., a lateral flow assay strip configured to detect one or more target nucleic acid sequences). The lateral flow assay strip may have a first end and a second end and may be configured to transport a fluidic sample from the first end to the second end. In some embodiments, the lateral flow assay strip comprises one or more materials that allow fluid transport (e.g., via capillary action). Non-limiting examples of suitable materials include polyethersulfone, cellulose, polycarbonate, nitrocellulose, cellulose acetate, sintered polyethylene, glass fibers, polyvinylidene fluoride, and charge-modified nylon. In some embodiments, the lateral flow assay strip comprises a plurality of pores and/or a plurality of fibers (e.g., woven or non-woven fibers). In some cases, pores and/or interstices between fibers may advantageously facilitate fluid transport (e.g., via capillary action).

In certain embodiments, the lateral flow assay strip comprises a plurality of sub-regions (e.g., a sample region, a particle conjugate region, a test region). In some embodiments, the plurality of sub-regions comprises a first sub-region where a fluid (e.g., a fluidic sample) can be introduced to the lateral flow assay strip. The first sub-region may be proximal to the first end of the lateral flow assay strip and may be referred to as a sample region or a sample pad. As discussed in further detail below, a fluidic sample introduced to the sample pad of a lateral flow assay strip may comprise an amplification product mixture comprising labeled target amplification products (i.e., amplified target nucleic acid sequences conjugated to one or more labels), labeled control amplification products (i.e., amplified control nucleic acid sequences conjugated to one or more labels), labeled unextended primers, and/or unlabeled primers. Amplified nucleic acid sequences may also be referred to as amplicons.

In some embodiments, the lateral flow assay strip comprises a second sub-region positioned between the first sub-region and a second end of the lateral flow assay strip. The second sub-region may be directly adjacent to the first sub-region or may be indirectly adjacent to the first sub-region (i.e., one or more intervening regions may be present between the first sub-region and the second sub-region). In some cases, the second sub-region may be referred to as a particle conjugate region or a particle conjugate pad. In certain embodiments, the second sub-region of the lateral flow assay strip comprises a plurality of labeled particles. In certain instances, the particles comprise gold nanoparticles (e.g., colloidal gold nanoparticles), latex particles (e.g., colored latex beads), silver particles (e.g., silver nanoparticles), magnetic particles, quantum dots, and carbon particles (e.g., carbon nanoparticles). The particles may be labeled with any suitable label. Non-limiting examples of suitable labels include biotin, streptavidin, fluorescein isothiocyanate (FITC), fluorescein amidite (FAM), fluorescein, digoxigenin (DIG), Texas Red, dinitrophenyl, tetramethylrhodamine (TAMRA), Dansyl, Cascade Blue, Cy5, a FLAG peptide (DYKDDDDK, SEQ ID NO: 1), a His peptide (HHHHHH, SEQ ID NO: 2), a HA peptide (YPYDVPDYA, SEQ ID NO: 3), and/or a Myc peptide (EQKLISEEDL, SEQ ID NO: 4). In certain embodiments, a fluidic sample transported through the second sub-region comprises labeled target amplification products and/or labeled control amplification products. In some such embodiments, labeled particles of the second sub-region may bind to the labeled target amplification products and/or labeled control amplification products.

In some embodiments, the lateral flow assay strip comprises a third sub-region positioned between the second sub-region and a second end of the lateral flow assay strip. The third sub-region may be directly adjacent to the second sub-region or may be indirectly adjacent to the second sub-region (i.e., one or more intervening regions may be present between the second sub-region and the third sub-region). In some cases, the third sub-region may be referred to as a test region or a test pad. In certain embodiments, the third sub-region of the lateral flow assay strip comprises one or more test lines. The one or more test lines may have any suitable shape or pattern (e.g., one or more straight lines, curved lines, dots, squares, check marks, x marks). In some embodiments, the one or more test lines are configured to indicate the presence or absence of at least one target nucleic acid sequence in a fluidic sample. In some embodiments, the one or more test lines comprise one or more capture reagents. The one or more capture reagents may, in some instances, comprise one or more biological capture reagents (e.g., immobilized antibodies). Non-limiting examples of suitable biological capture reagents include anti-DIG antibodies, anti-FITC antibodies, anti-FAM antibodies, anti-fluorescein antibodies, anti-biotin antibodies, anti-streptavidin antibodies, anti-Texas Red antibodies, anti-TAMRA antibodies, anti-dinitrophenyl antibodies, anti-Dansyl antibodies, anti-Cascade Blue antibodies, anti-Cy5 antibodies, anti-FLAG peptide antibodies, anti-His peptide antibodies, anti-HA peptide antibodies, anti-Myc peptide antibodies, biotin, and streptavidin. In certain embodiments, a fluidic sample transported through the third sub-region comprises labeled target amplification products and/or labeled control amplification products. In some such embodiments, one or more capture reagents (e.g., biological capture reagents) of one or more test lines bind to the labeled target amplification products.

In some embodiments, the third sub-region of the lateral flow assay strip comprises two or more test lines. In some instances, the two or more test lines comprise a first test line configured to detect a first target nucleic acid sequence and a second test line configured to detect a second target nucleic acid sequence. In certain embodiments, the first target nucleic acid sequence is different from the second target nucleic acid sequence. In certain embodiments, the first target nucleic acid sequence is the same as the second target nucleic acid sequence. In some embodiments, each test line of the two or more test lines is configured to detect a different target nucleic acid sequence.

In some embodiments, the third sub-region of the lateral flow assay strip comprises one or more control lines. The control line(s) may have any suitable shape or pattern (e.g., one or more straight lines, curved lines, dots, squares, check marks, x marks). In some embodiments, a first control line is a flow control line. In some cases, a visible change of the flow control line (e.g., a change from a first color to a second color, an opaque band becoming more or less visible) indicates that a liquid was successfully transported through the lateral flow assay strip. In some embodiments, the flow control line comprises a non-biological fluid indicator. As used herein, a non-biological fluid indicator refers to a fluid indicator that does not comprise a biological capture reagent (e.g., an antibody). According to some embodiments, the non-biological fluid indicator comprises a pH-sensitive material, a moisture-sensitive material, and/or a chemically-sensitive material.

In some embodiments, the non-biological fluid indicator comprises a pH-sensitive material. A pH-sensitive material generally refers to a material that exhibits a visible change (e.g., a change from a first color to a second color, an opaque band becoming more or less visible) upon exposure to a certain pH and/or a change in pH. In some embodiments, the pH-sensitive material comprises litmus paper. Litmus paper may comprise one or more dyes extracted from lichens (e.g., Roccella tinctoria). In some embodiments, the pH-sensitive material comprises litmus, phenolphthalein, and/or phenol red.

In some embodiments, the non-biological fluid indicator comprises a moisture-sensitive material (e.g., a hydrochromic material). A moisture-sensitive material generally refers to a material that exhibits a visible change (e.g., a change from a first color to a second color, an opaque band becoming more or less visible) upon exposure to moisture. Non-limiting examples of suitable moisture-sensitive materials include cobalt (II) chloride and copper (II) chloride (CuCl₂). As a non-limiting embodiment, cobalt (II) chloride (CoCl₂) turns from blue to pink, and copper (II) chloride turns from yellow to blue. In some embodiments, a moisture-sensitive material comprises manganese. In some embodiments, a moisture-sensitive material comprises betaine dye. In some embodiments, a moisture-sensitive material becomes transparent upon exposure to moisture.

In some embodiments, the non-biological fluid indicator comprises a chemically-sensitive material. In some embodiments, the chemically-sensitive material exhibits a visible change (e.g., a change from a first color to a second color, an opaque band becoming more or less visible) upon exposure to one or more salts, nucleic acids, or proteins. In certain embodiments, the chemically-sensitive material is photochromic, thermochromic, and/or electrochromic. Non-limiting examples of suitable chemically-sensitive materials include 3, 3′, 5, 5′-tetramethylbenzidine (“TMB”) and peroxidase fluorogenic substrates.

In some embodiments, the non-biological fluid indicator comprises a dye configured to be transported through a lateral flow assay strip (e.g., via capillary action) with flow of a fluidic sample. In some such embodiments, the dye may exhibit a visible change (e.g., movement from one location of a lateral flow assay strip to another location of the lateral flow assay strip) upon contact with a fluidic sample flowing through the lateral flow assay strip. In certain embodiments, the dye comprises a food colorant or other color additive. Examples of suitable dyes include, but are not limited to, FD&C Blue No. 1 (Brilliant Blue FCF, E133), FD&C Blue No. 2 (Indigo Carmine, E132), FD&C Green No. 3 (Fast Green FCF, E143), FD&C Red No. 3 (Erythrosine, E127), FD&C Red No. 40 (Allura Red AC, E129), FD&C Yellow No. 5 (Tartrazine, E102), FD&C Yellow No. 6 (Sunset Yellow FCF, E110), Citrus Red 2, and Orange B.

In some cases, a flow control line comprising a non-biological fluid indicator results in a visible line (or other marking) that is brighter (e.g., more intense, more easily visible to an unaided eye) upon contact with a liquid than conventional flow control lines comprising a biological fluid indicator. In some embodiments, visible lines (or other markings) produced using flow control lines comprising a non-biological fluid indicator are at least 10%, at least 20%, at least 30%, at least 40%, at least 50%, at least 60%, at least 70%, at least 80%, at least 90%, at least 95%, or at least 100% darker than visible lines (or other markings) produced using flow control lines comprising a biological fluid indicator. In some cases, the darkness of visible lines (or other markings) may be measured and compared by comparing the pixel intensities of the visible lines (or other markings) in one or more images obtained from a camera.

Some aspects of the disclosure relate to lateral flow assay strips, diagnostic systems, and diagnostic methods that produce fewer false negative results than lateral flow assay strips, diagnostic systems, or diagnostic methods using a flow control line comprising a biological fluid indicator. In some embodiments, lateral flow assay strips, diagnostic systems, and/or diagnostic methods using a flow control line comprising a non-biological fluid indicator produce at least 10%, at least 20%, at least 30%, at least 40%, at least 50%, at least 60%, at least 70%, at least 80%, at least 90%, at least 95%, or at least 100% fewer false negative results than otherwise similar lateral flow assay strips, diagnostic systems, and/or diagnostic methods using a flow control line comprising a biological fluid indicator.

In some embodiments, the one or more control lines comprise a nucleic acid control line. In some embodiments, for example, the nucleic acid control line is configured to detect a nucleic acid sequence that is generally present in one or more target species (e.g., a species of a subject from whom a sample was obtained). The one or more target species may comprise humans and/or an animal species. In some cases, a visible change of the nucleic acid control line (e.g., a change from a first color to a second color, an opaque band becoming visible) indicates that a sample from the target species was successfully collected, nucleic acids from the sample were amplified, and the control amplification products were transported through the lateral flow assay strip.

In some embodiments, the lateral flow assay strip comprises two or more control lines. In some instances, for example, the lateral flow assay strip comprises one or more nucleic acid control lines and one or more flow control lines (e.g., one or more flow control lines comprising a non-biological fluid indicator).

In certain embodiments, the lateral flow assay strip comprises a fourth sub-region positioned between the third sub-region and a second end of the lateral flow assay strip. The fourth sub-region may be directly adjacent to the third sub-region or may be indirectly adjacent to the third sub-region (i.e., one or more intervening regions may be present between the third sub-region and the fourth sub-region). In some cases, the fourth sub-region may be referred to as a wicking region or a wicking pad. The fourth sub-region may be configured to absorb fluid flowing through the lateral flow assay strip.

As a non-limiting, illustrative example, a fluidic sample comprising a target amplification product (e.g., a SARS-CoV-2 amplicon) labeled with a first label (e.g., biotin) and a second label (e.g., FAM) and a control amplification product (e.g., a RNaseP amplicon) labeled with a first label (e.g., biotin) and a second label (e.g., DIG) may be introduced to the first sub-region (e.g., the sample pad) of a lateral flow assay strip. In some cases, as the labeled target amplification product and the labeled control amplification product are transported through the second sub-region (e.g., the particle conjugate pad), a first streptavidin-labeled gold nanoparticle binds to the biotin label of the labeled target amplification product and a second streptavidin-labeled gold nanoparticle binds to the biotin label of the labeled control amplification product. In some cases, the gold nanoparticle-amplicon conjugates are transported through the third sub-region (e.g., the test pad), which comprises a first test line comprising an anti-FAM antibody, a nucleic acid control line comprising an anti-DIG antibody, and a flow control line comprising a non-biological fluid indicator. In some embodiments, the gold nanoparticle-target amplicon conjugate may be captured by the anti-FAM antibody, and an opaque band may develop at the first test line as additional gold nanoparticle-target amplicon conjugates are captured by the anti-FAM antibodies of the first test line. In some embodiments, the gold nanoparticle-control amplicon conjugate may be captured by the anti-DIG antibody, and an opaque band may develop at the nucleic acid control line as additional gold nanoparticle-control amplicon conjugates are captured by the anti-DIG antibodies of the nucleic acid control line. In some cases, the fluidic sample may be further transported to the flow control line, and the non-biological fluid indicator of the flow control line may provide visual indication that a liquid reached the flow control line. In some cases, the fluidic sample may be further transported to the fourth sub-region (e.g., the wicking pad) of the lateral flow assay strip.

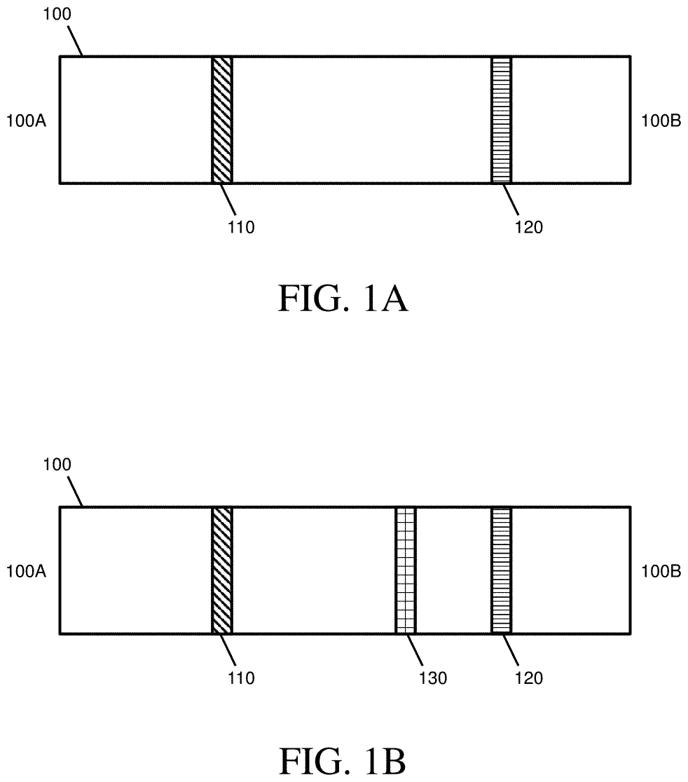

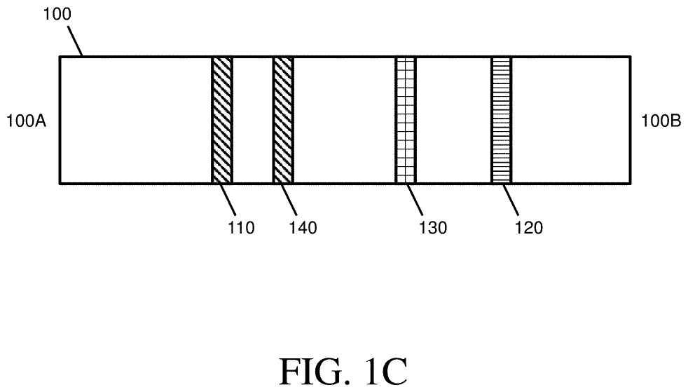

FIGS. 1A-1C illustrate non-limiting examples of a lateral flow assay strip. In FIG. 1A, lateral flow assay strip 100 having first end 100A and second end 100B comprises first test line 110 and flow control line 120. As shown in FIG. 1A, flow control line 120 is positioned between first test line 110 and second end 100B.

In some embodiments, first test line 110 is configured to detect a first target nucleic acid sequence. The first target nucleic acid sequence may, for example, be a nucleic acid sequence of a pathogen (e.g., a coronavirus such as SARS-CoV-2 or a variant thereof, an influenza virus). In some embodiments, the first test line comprises one or more capture reagents (e.g., one or more immobilized antibodies). In some embodiments, flow control line 120 is configured to exhibit a visible change upon contact with a liquid. In some embodiments, flow control line 120 comprises a non-biological fluid indicator (e.g., a pH-sensitive material, a moisture-sensitive material, and/or a chemically-sensitive material).

In operation, a fluidic sample is introduced at first end 100A of lateral flow assay strip 100. The fluidic sample subsequently flows through lateral flow assay strip 100 (e.g., via capillary action) to first test line 110. The fluidic sample then continues to flow through lateral flow assay strip 100 to flow control line 120. If the fluidic sample successfully reaches flow control line 120, flow control line 120 undergoes a visible change (e.g., a change from a first color to a second color, an opaque band becoming visible).

FIG. 1B illustrates a non-limiting example where lateral flow assay strip 100 further comprises a second control line 130 positioned between first test line 110 and first control line 120. In some embodiments, second control line 130 is a nucleic acid control line. FIG. 1C illustrates a non-limiting example where lateral flow assay strip 100 further comprises a second test line 140 positioned between first test line 110 and second control line 130. In some cases, second test line 140 is configured to detect a second target nucleic acid sequence that is different from the first target nucleic acid sequence.

The lateral flow assay strip may have any suitable dimensions. In some embodiments, the lateral flow assay strip has a relatively short length (i.e., the longest dimension of the lateral flow assay strip). In certain embodiments, the lateral flow assay strip has a length of 25 cm or less, 20 cm or less, 15 cm or less, 10 cm or less, 5 cm or less, or 2 cm or less. In certain embodiments, the lateral flow assay strip has a length in a range from 2 cm to 5 cm, 2 cm to 10 cm, 2 cm to 15 cm, 2 cm to 20 cm, 2 cm to 25 cm, 5 cm to 10 cm, 5 cm to 15 cm, 5 cm to 20 cm, 5 cm to 25 cm, 10 cm to 15 cm, 10 cm to 20 cm, 10 cm to 25 cm, 15 cm to 20 cm, 15 cm to 25 cm, or 20 cm to 25 cm.

In some embodiments, the lateral flow assay strip has a relatively narrow maximum width. In certain embodiments, the lateral flow assay strip has a maximum width of 20 mm or less, 15 mm or less, 12 mm or less, 10 mm or less, 9 mm or less, 8 mm or less, 7 mm or less, 6 mm or less, 5 mm or less, 4 mm or less, 3 mm or less, 2 mm or less, or 1 mm or less. In some embodiments, the lateral flow assay strip has a maximum width in a range from 1 mm to 5 mm, 1 mm to 10 mm, 1 mm to 12 mm, 1 mm to 15 mm, 1 mm to 20 mm, 5 mm to 10 mm, 5 mm to 12 mm, 5 mm to 15 mm, 5 mm to 20 mm, 10 mm to 15 mm, 10 mm to 20 mm, or 15 mm to 20 mm.

In some embodiments, the lateral flow assay strip is relatively thin. In certain embodiments, the lateral flow assay strip has a maximum thickness of 5 mm or less, 4 mm or less, 3 mm or less, 2 mm or less, 1 mm or less, 0.9 mm or less, 0.8 mm or less, 0.7 mm or less, 0.6 mm or less, 0.5 mm or less, 0.4 mm or less, 0.3 mm or less, 0.2 mm or less, or 0.1 mm or less. In some embodiments, the lateral flow assay strip has a maximum thickness in a range from 0.1 mm to 0.2 mm, 0.1 mm to 0.3 mm, 0.1 mm to 0.4 mm, 0.1 mm to 0.5 mm, 0.1 mm to 0.6 mm, 0.1 mm to 0.7 mm, 0.1 mm to 0.8 mm, 0.1 mm to 0.9 mm, 0.1 mm to 1 mm, 0.1 mm to 2 mm, 0.1 mm to 5 mm, 0.2 mm to 0.4 mm, 0.2 mm to 0.5 mm, 0.2 mm to 0.6 mm, 0.2 mm to 0.7 mm, 0.2 mm to 0.8 mm, 0.2 mm to 0.9 mm, 0.2 mm to 1 mm, 0.2 mm to 2 mm, 0.2 mm to 5 mm, 0.5 mm to 1 mm, 0.5 mm to 2 mm, 0.5 mm to 5 mm, 1 mm to 2 mm, or 1 mm to 5 mm.

### Diagnostic Systems & Methods

Lateral flow assay strips described herein may be used in a diagnostic system or method to detect the presence of one or more target nucleic acid sequences (e.g., one or more sequences from nucleic acids of one or more target pathogens). In some embodiments, the diagnostic system comprises one or more sample-collecting components (e.g., swabs, cell scrapers, tongue depressors) configured to facilitate collection of a biological sample from a subject. The diagnostic system may also comprise one or more reagents configured to facilitate cell lysis and/or nucleic acid amplification (e.g., amplification of a target nucleic acid sequence in a biological sample). In some cases, the diagnostic system further comprises a diagnostic apparatus (e.g., a “chimney” type apparatus, a cartridge apparatus, a blister pack apparatus, an integrated swab apparatus) comprising a lateral flow assay strip (e.g., a lateral flow assay strip comprising one or more test lines configured to detect one or more target nucleic acid sequences and a flow control line comprising a non-biological fluid indicator). The diagnostic system may also comprise reaction vessels (e.g., reaction tubes, reaction vials, and the like, which may be used to carry out a reaction involving a sample and/or a reagent) and/or heaters.

Unlike prior art diagnostic testing schemes, the diagnostic system may not require knowledge of even basic laboratory techniques or chemistry or biology. Additionally, unlike diagnostic testing schemes that require bulky equipment, the diagnostic system may be easily transported and/or easily stored in homes, businesses, schools, and other non-laboratory settings. In some embodiments, the components of the diagnostic system, including reagents, may be stored under ambient conditions (e.g., room temperature and atmospheric pressure).

Diagnostic system components and methods of using the diagnostic system to detect one or more target nucleic acid sequences are described below.

### Biological Samples

Aspects of the disclosure relate to compositions and methods for detecting one or more target nucleic acid sequences in a biological sample. In some embodiments, the biological sample is obtained from a subject (e.g., a human subject, an animal subject). The subject may be any mammal, such as a human, non-human primate (e.g., monkey, chimpanzee, gorilla, orangutan, etc.), dog, cat, pig, horse, hamster, guinea pig, rat, mouse, etc. In certain embodiments, the subject is a human. The biological sample, in some embodiments, is collected from a subject who is suspected of having the disease(s) the test screens for, such as a coronavirus (e.g., COVID-19) and/or influenza (e.g., influenza type A or influenza type B).

Exemplary biological samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In certain embodiments, the biological sample comprises a nasal secretion. The nasal secretion may be collected by a nasal swab. In some instances, for example, the sample is an anterior nares specimen. In certain embodiments, the sample comprises an oral secretion (e.g., saliva). The oral secretion may be deposited directly into a reaction tube.

In some embodiments, a biological sample obtained from a subject is a fluidic sample (e.g., a sample comprising one or more fluids). In some embodiments, a biological sample obtained from a subject is combined with one or more fluids (e.g., one or more buffers) to form a fluidic sample. Non-limiting examples of suitable fluids include phosphate-buffered saline (PBS) and Tris.

### Target Nucleic Acid Sequences

Lateral flow assay strips, diagnostic systems, and/or diagnostic methods presented herein, in some embodiments, may be used to detect the presence of one or more target nucleic acid sequences. The one or more target nucleic acid sequences may be associated with a variety of diseases or disorders, as described below. In some embodiments, the lateral flow assay strips, diagnostic systems, and/or diagnostic methods may be used to diagnose at least one disease or disorder caused by a pathogen (e.g., a human pathogen, an animal pathogen).

In some embodiments, the one or more target nucleic acid sequences comprise a nucleic acid sequence of a virus (e.g., a viral pathogen). Non-limiting examples of viruses include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the one or more target nucleic acid sequences comprise a sequence of SARS-CoV-2 or a variant thereof. In certain embodiments, the one or more target nucleic acid sequences comprise a nucleic acid sequence of a SARS-CoV-2 variant comprising one or more mutations. In some embodiments, the one or more mutations comprise a D614G, A222V, N501Y, E484K, K417N, K417T, S494P, A570D, P681H, T716I, S982A, D1118H, K1191N, D80A, D215G, A701V, T19R, V70F, T95I, G142D, R158G, W258L, L452R, T478K, P681R, D950N, L18F, T20N, P26S, D138Y, R1905, H655Y, T1027I, S13I, W152C, LSF, D80G, F157S, D253G, S477N, T859N, D950H, Q957R, E154K, E484Q, and/or Q1071H mutation. In some embodiments, the SARS-CoV-2 variant is SARS-CoV-2 D614G, a SARS-CoV-2 variant of B.1.1.7 lineage (e.g., 201/501Y.V1 Variant of Concern (VOC) 202012/01), a SARS-CoV-2 variant of B.1.351 lineage (e.g., 20H/501.V2), a SARS-CoV-2 variant of B.1.617.2 lineage (e.g., 21A/S:478K), a SARS-CoV-2 variant of P.1 lineage (e.g., 20J/501Y.V3), a SARS-CoV-2 variant of B.1.427 lineage (e.g., 20C/S:452R), a SARS-CoV-2 variant of B.1.429 lineage (e.g., 20C/S:452R), a SARS-CoV-2 variant of B.1.525 lineage (e.g., 20A/S:484K), a SARS-CoV-2 variant of B.1.526 lineage (e.g., 20C/S:484K), a SARS-CoV-2 variant of B.1.617.1 lineage (e.g., 21A/S:154K), or a SARS-CoV-2 variant of B.1.617.3 lineage (e.g., 20A). In some embodiments, the one or more target nucleic acid sequences comprise a nucleic acid sequence of an influenza virus. The influenza virus may be an influenza A virus (e.g., H1N1, H3N2) or an influenza B virus.

In some embodiments, the one or more target nucleic acid sequences comprise a nucleic acid sequence of a bacterium (e.g., a bacterial pathogen), a fungus (e.g., a fungal pathogen), or a protozoa (e.g., a protozoan pathogen).

In some embodiments, the one or more target nucleic acid sequences comprise a nucleic acid sequence of a cancer cell. Cancer cells generally have unique mutations found in tumor cells and absent in normal cells. In some embodiments, the lateral flow assay strips, systems, and/or methods described herein are configured to examine a subject's predisposition to certain types of cancer based on specific genetic mutations. In some embodiments, the lateral flow assay strips, systems, and/or methods are configured to detect one or more target nucleic acid sequences associated with a genetic disorder.

In some embodiments, the lateral flow assay strips, diagnostic systems, and/or diagnostic methods are configured to detect one or more target nucleic acid sequences associated with a single nucleotide polymorphism (SNP). In some such embodiments, the lateral flow strips, diagnostic systems, and/or diagnostic methods described herein may be used for rapid genotyping to detect the presence or absence of a SNP, which may affect medical treatment.

According to some embodiments, lateral flow assay strips, diagnostic systems, and/or diagnostic methods described herein may be configured to detect two or more nucleic acid sequences. In certain cases, for example, a lateral flow assay strip may comprise a first test line configured to detect a first target nucleic acid sequence and a second test line configured to detect a second target nucleic acid sequence. In some embodiments, the first target nucleic acid sequence is a nucleic acid sequence of SARS-CoV-2 and the second target nucleic acid sequence is a nucleic acid sequence of a SARS-CoV-2 variant. In some embodiments, the first target nucleic acid sequence is a nucleic acid sequence of SARS-CoV-2 or a variant thereof and the second target nucleic acid sequence is a nucleic acid sequence of an influenza virus. In some embodiments, the first target nucleic acid sequence is a nucleic acid sequence of a virus and the second target nucleic acid sequence is a nucleic acid sequence of a bacterium. In certain embodiments, the lateral flow assay strips, diagnostic systems, and/or diagnostic methods are configured to detect 2 or more, 3 or more, 4 or more, 5 or more, 6 or more, 7 or more, 8 or more, 9 or more, or 10 or more target nucleic acid sequences.

### Control Nucleic Acid Sequences

In some embodiments, lateral flow assay strips, diagnostic methods, and diagnostic systems described herein comprise (or use) primers designed to amplify a human or animal nucleic acid sequence that is not associated with a target nucleic acid sequence from a pathogen, a cancer cell, or a contaminant. In some such embodiments, the human or animal nucleic acid sequence may act as a control and is referred to as a “control nucleic acid sequence.” For example, successful amplification and detection of a control nucleic acid sequence may indicate that a sample was properly collected and the diagnostic test was properly run (e.g., an amplification reaction was successful).

A control nucleic acid sequence is typically a gene or portion of a gene that is widely expressed and/or expressed at a high level in a target species (e.g., a human or other mammal). Such genes are often referred to as “housekeeping genes.” Examples of housekeeping genes include, but are not limited to, RNase P, GAPDH, B2M, ACTB, POLR2A, UBC, PPIA, HPRT1, GUSB, TBP, and H3F3A. In some embodiments, a control nucleic acid sequence encodes at least a portion of a gene selected from RNase P, GAPDH, B2M, ACTB, POLR2A, UBC, PPIA, HPRT1, GUSB, TBP, H3F3A, POLR2A, RPLPO, L19, B2M, RPS17, ALAS1, CD74, CK18, HMBS, IPO8, PGK1, YWHAZ, and STATH.

### Cell Lysis

In some embodiments, lysis may be performed on a sample by chemical lysis (e.g., exposing the sample to one or more lysis reagents) and/or thermal lysis (e.g., heating the sample). In chemical lysis, lysis may be performed by one or more lysis reagents. A lysis reagent may refer generally to a reagent that promotes cell lysis either alone or in combination with one or more other reagents and/or one or more conditions (e.g., heating). In some embodiments, the one or more lysis reagents comprise one or more enzymes. Non-limiting examples of suitable enzymes include lysozyme, lysostaphin, zymolase, cellulase, protease, and glycanase. In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40.

In some embodiments, the one or more lysis reagents comprise an RNase inhibitor (e.g., a murine RNase inhibitor). In some embodiments, the RNase inhibitor concentration is at least 0.1 U/μL, at least 1.0 U/μL, or at least 2.0 U/μL. In certain embodiments, the RNase inhibitor concentration is in a range from 0.1 U/μL to 0.5 U/μL, 0.1 U/μL to 1.5 U/μL, or 1.0 U/μL to 2.0 U/μL. In some embodiments, the one or more lysis reagents comprise Tween (e.g., Tween 20, Tween 80).

In some embodiments, at least one of the one or more lysis reagents may be in solid form (e.g., lyophilized, dried, crystallized, air jetted, etc.). In some cases, all of the one or more lysis reagents may be in solid form (e.g., lyophilized, dried, crystallized, air jetted, etc.). In certain embodiments, one or more solid lysis reagents may be in the form of a lysis pellet, capsule, gelcap, or tablet. The lysis pellet, capsule, gelcap, or tablet may comprise any lysis reagent described herein. In certain embodiments, the lysis pellet, capsule, gelcap, tablet may comprise one or more additional reagents (e.g., reagents to reduce or eliminate cross contamination). In a particular, non-limiting embodiment, the lysis pellet, capsule, gelcap or tablet may comprise Thermolabile Uracil-DNA Glycosylase (UDG) (e.g., at a concentration of about 0.02 U/uL) and murine RNAse inhibitor (e.g., at a concentration of about 1 U/uL).

As noted above, thermal lysis may be accomplished by applying heat to the sample. In some embodiments, thermal lysis may be performed by applying a lysis heating protocol comprising heating the sample at one or more temperatures for one or more time periods or durations using any suitable heater. In certain instances, the one or more temperatures comprise a temperature of at least 37° C., at least 40° C., at least 50° C., at least 60° C., at least 63.5° C., at least 65° C., at least 70° C., at least 80° C., or at least 90° C. In certain instances, the one or more temperatures comprise a temperature in a range from 37° C. to 50° C., 37° C. to 60° C., 37° C. to 63.5° C., 37° C. to 65° C., 37° C. to 70° C., 37° C. to 80° C., 37° C. to 90° C., 50° C. to 60° C., 50° C. to 63.5° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the one or more time periods comprise a time period of at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 50 minutes, at least 55 minutes, or at least 60 minutes. In certain instances, the one or more time periods comprise a time period in a range from 1 to 3 minutes, 1 to 5 minutes, 1 to 10 minutes, 1 to 15 minutes, 1 to 30 minutes, 1 to 40 minutes, 1 to 50 minutes, 1 to 60 minutes, 3 to 5 minutes, 3 to 10 minutes, 3 to 20 minutes, 3 to 30 minutes, 3 to 40 minutes, 3 to 50 minutes, 3 to 60 minutes, 5 to 20 minutes, 5 to 30 minutes, 5 to 40 minutes, 5 to 50 minutes, 5 to 60 minutes, 10 to 30 minutes, 10 to 40 minutes, 10 to 50 minutes, 10 to 60 minutes, 20 to 40 minutes, 20 to 50 minutes, 20 to 60 minutes, 30 to 50 minutes, 30 to 60 minutes, 40 to 60 minutes, or 50 to 60 minutes.

### Nucleic Acid Amplification

Following lysis, one or more target nucleic acid sequences (e.g., a nucleic acid sequence of a target pathogen) and/or control nucleic acid sequences may be amplified, according to some embodiments. Aspects of the disclosure relate to lateral flow assay strips, diagnostic systems, and diagnostic methods for detecting target nucleic acid sequences and/or control nucleic acid sequences in a biological sample. In some embodiments, the diagnostic methods comprise a step of performing a nucleic acid amplification reaction (e.g., an isothermal amplification reaction) on a biological sample in order to amplify a target nucleic acid sequence and/or a control nucleic acid sequence.

In some cases, a target pathogen may have RNA as its genetic material. In certain instances, for example, a target pathogen may be an RNA virus (e.g., a coronavirus, an influenza virus). In some such cases, the target pathogen's RNA may need to be reverse transcribed to DNA prior to amplification.

In some embodiments, reverse transcription may be performed by exposing lysate (i.e., product(s) of lysis) to one or more reverse transcription reagents. In certain instances, the one or more reverse transcription reagents comprise a reverse transcriptase, a DNA-dependent polymerase, and/or a ribonuclease (RNase). A reverse transcriptase may refer generally to an enzyme that transcribes RNA to complementary DNA (cDNA) by polymerizing deoxyribonucleotide triphosphates (dNTPs). In some embodiments, a reverse transcriptase is selected from the group consisting of HIV-1 reverse transcriptase, Moloney murine leukemia virus (M-MLV) reverse transcriptase, and avian myeloblastosis virus (AMV) reverse transcriptase. An RNase generally refers to an enzyme that catalyzes the degradation of RNA. In some cases, an RNase may be used to digest RNA from an RNA-DNA hybrid.

In some embodiments, DNA may be amplified according to any nucleic acid amplification method known in the art. In some embodiments, the nucleic acid amplification method is an isothermal amplification method. In some cases, the isothermal nucleic acid amplification method, unlike PCR-based methods, avoids use of expensive, bulky laboratory equipment for precise thermal cycling. Isothermal amplification methods include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method is LAMP. LAMP refers to a method of amplifying a target nucleic acid using at least four primers through the creation of a series of stem-loop structures. Due to its use of multiple primers, LAMP may be highly specific for a target nucleic acid sequence.

In some cases, at least one of the one or more amplification reagents is in solid form (e.g., lyophilized, dried, crystallized, air jetted). In some cases, all of the one or more amplification reagents are in solid form (e.g., lyophilized, dried, crystallized, air jetted). In certain embodiments, one or more amplification reagents are in the form of an amplification pellet or tablet. The amplification pellet or tablet may comprise any amplification reagent described herein.

In some embodiments, an isothermal amplification method described herein comprises applying heat to a sample according to an amplification heating protocol. In certain instances, the amplification heating protocol comprising heating the sample at one or more temperatures for one or more time periods using any heater described herein. However, other embodiments of the present invention do not require a step of applying heat to a sample. In such embodiments, the step of applying an amplification heating protocol as described below would not be necessary for nucleic acid amplification, and would not be performed.

In some embodiments, an amplification heating protocol comprises heating the sample at one or more temperatures for one or more time periods (e.g., 1, 2, 3, 4, 5, or more time periods). In certain instances, the one or more temperatures comprise a temperature of at least 37° C., at least 40° C., at least 50° C., at least 60° C., at least 63.5° C., at least 65° C., at least 70° C., at least 80° C., or at least 90° C. In certain instances, the one or more temperatures comprise a temperature in a range from 37° C. to 50° C., 37° C. to 60° C., 37° C. to 63.5° C., 37° C. to 65° C., 37° C. to 70° C., 37° C. to 80° C., 37° C. to 90° C., 50° C. to 60° C., 50° C. to 63.5° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the one or more time periods comprise a time period of at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 50 minutes, at least 55 minutes, or at least 60 minutes. In certain instances, the one or more time periods comprise a time period in a range from 1 to 3 minutes, 1 to 5 minutes, 1 to 10 minutes, 1 to 15 minutes, 1 to 30 minutes, 1 to 40 minutes, 1 to 50 minutes, 1 to 60 minutes, 3 to 5 minutes, 3 to 10 minutes, 3 to 20 minutes, 3 to 30 minutes, 3 to 40 minutes, 3 to 50 minutes, 3 to 60 minutes, 5 to 20 minutes, 5 to 30 minutes, 5 to 40 minutes, 5 to 50 minutes, 5 to 60 minutes, 10 to 30 minutes, 10 to 40 minutes, 10 to 50 minutes, 10 to 60 minutes, 20 to 40 minutes, 20 to 50 minutes, 20 to 60 minutes, 30 to 50 minutes, 30 to 60 minutes, 40 to 60 minutes, or 50 to 60 minutes.

In an illustrative, non-limiting embodiment, a lysis and/or amplification heating protocol may comprise heating a sample at 37° C. for 3 minutes, subsequently heating the sample at 63.5° C. for 40 minutes, and subsequently decreasing the temperature to 37° C.

### Molecular Switches

As described herein, a sample may undergo lysis and amplification prior to detection. Reagents associated with lysis and/or amplification may be in solid form (e.g., lyophilized, dried, crystallized, air jetted, etc.). In certain embodiments, one or more (and, in some cases, all) of the reagents necessary for lysis and/or amplification may be present in a single pellet, capsule, gelcap, or tablet. In some embodiments, the pellet, capsule, gelcap, or tablet may comprise two or more enzymes, and it may be necessary for the enzymes to be activated in a particular order. Therefore, in some embodiments, the enzyme-containing tablet, pellet, capsule, or gelcap may further comprise one or more molecular switches.

Molecular switches, as used or described herein, may be molecules that, in response to certain conditions, reversibly switch between two or more stable states. In some embodiments, a condition that causes the molecular switch to change its configuration may be associated with any one or any combination of: pH, light, temperature, an electric current, microenvironment, and presence of ions and/or other ligands. In one embodiment, the condition may be heat. In some embodiments, the molecular switches described herein may be aptamers. Aptamers may refer generally to oligonucleotides or peptides that may bind to specific target molecules (e.g., the enzymes described herein). The aptamers, upon exposure to heat or other conditions, may dissociate from the enzymes. With use of molecular switches, one or more of the processes described herein (e.g., lysis, decontamination, reverse transcription, and amplification, etc.) may be performed in a single test tube with a single enzymatic tablet, pellet, capsule, or gelcap.

### CRISPR/Cas

In some embodiments, CRISPR/Cas detection techniques may be used to detect a target nucleic acid sequence. For example, one or more CRISPR/Cas detection reagents may be included on a lateral flow assay strip. CRISPR generally may refer to Clustered Regularly Interspaced Short Palindromic Repeats, and Cas generally may refer to a particular family of proteins. In some embodiments, the CRISPR/Cas detection platform or techniques may be combined with an isothermal amplification method to create a single-step reaction (Joung et al., “Point-of-care testing for COVID-19 using SHERLOCK diagnostics,” 2020). For example, amplification and CRISPR detection may be performed using reagents having compatible chemistries (e.g., reagents that do not interact detrimentally with one another and are sufficiently active to perform amplification and detection). In some embodiments, CRISPR/Cas detection may be combined with LAMP.

### Additional Reagents

In some embodiments, a diagnostic system comprise one or more additional reagents. In certain embodiments, the one or more reagents comprise one or more reagents to reduce or eliminate potential carryover contamination from prior tests (e.g., prior tests conducted with a common apparatus and/or in a same area). In some embodiments, the one or more reagents comprise thermolabile uracil DNA glycosylase (UDG). UDG may, in some instances, prevent carryover contamination from prior tests by degrading products that have already been amplified (i.e., amplicons) while leaving unamplified samples untouched and ready for amplification. In some embodiments, a concentration of UDG may be at least 0.01 U/μL, at least 0.03 U/μL, or at least 0.05 U/μL. In certain embodiments, the concentration of UDG may be in a range from 0.01 U/μL to 0.02 U/μL or 0.01 U/μL to 0.04 U/μL.

In some embodiments, the one or more reagents comprise one or more additives that may enhance reagent stability (e.g., protein stability). Non-limiting examples of suitable additives include trehalose, polyethylene glycol (PEG), polyvinyl alcohol (PVA), and glycerol.

In some embodiments, the one or more reagents comprise one or more reaction buffers. Non-limiting examples of suitable buffers include phosphate-buffered saline (PBS) and Tris. In some embodiments, the one or more buffers have a relatively neutral pH. In some embodiments, the one or more buffers have a pH in a range from 5.0 to 7.0, 6.0 to 8.0, 7.0 to 9.0, or 8.0 to 9.0. In some embodiments, the one or more buffers comprise one or more salts. Non-limiting examples of suitable salts may include magnesium acetate tetrahydrate, potassium acetate, and potassium chloride.

### Shelf Stability

The shelf stability of reagents, liquids (e.g., buffers), and other chemical materials used in the diagnostic systems and methods described herein, as well as the stability of the equipment that may be used to perform the diagnostic methods (for example, diagnostic apparatuses (e.g., a “chimney” type diagnostic apparatus, a cartridge diagnostic apparatus, a blister pack diagnostic apparatus, an integrated swab diagnostic apparatus); component(s) usable by or in the diagnostic apparatuses (e.g., a sample-collecting component such as a sample swab, etc.); device(s) used in or with the diagnostic apparatuses (e.g., heater(s), reactant carrier(s), reaction tube(s) and/or other reaction vessel(s), etc.); reagent(s) (e.g., lysis reagent(s), nucleic acid amplification reagent(s), CRISPR/Cas detection reagent(s), buffer(s), etc., any one or more of which may be provided in reagent carrier(s) and/or reaction vessel(s)); test component(s) used in rapid-diagnostic test procedures (e.g., lateral flow assay strip(s)); and reader device(s) used to read test results (e.g., sample readouts (e.g., photographs, illustrations, etc.) for a user to compare with actual test results to determine a presence or an absence of pathogen(s), application(s) installable on electronic devices to electronically read digital images of test results, etc.)) enable the equipment and the reagents, buffers, and other chemical materials to be transported and/or stored without the need for special transportation and/or storage considerations (e.g., a thermally controlled environment, a barometrically controlled environment, etc.).

In certain embodiments, one or more lysis reagents and/or amplification reagents may be in the form of one or more pellets, capsules, gelcaps, and/or tablets. In some embodiments, the one or more pellets, capsules, gelcaps, and/or tablets may be shelf stable for a relatively long period of time. In certain embodiments, the one or more pellets, capsules, gelcaps, and/or tablets may be shelf stable for at least 1 month, at least 3 months, at least 6 months, at least 1 year, at least 5 years, or at least 10 years. In certain embodiments, the one or more pellets, capsules, gelcaps, and/or tablets may be shelf stable for a time period in a range from 1 to 3 months, 1 to 6 months, 1 month to 1 year, 1 month to 5 years, 1 month to 10 years, 6 months to 1 year, 6 months to 5 years, 6 months to 10 years, 1 to 5 years, 1 to 10 years, or 5 to 10 years. In some embodiments, the one or more pellets, capsules, gelcaps, and/or tablets may be thermostabilized and may be stable across a wide range of temperatures. In some embodiments, the one or more pellets, capsules, gelcaps, and/or tablets may be stable at a temperature of at least 0° C., at least 10° C., at least 20° C., at least 25° C., at least 37° C., at least 65° C., at least 90° C., or at least 100° C. In some embodiments, the one or more pellets, capsules, gelcaps, and/or tablets may be stable at a temperature in a range from 0° C. to 20° C., 0° C. to 25° C., 0° C. to 37° C., 0° C. to 65° C., 0° C. to 90° C., 0° C. to 100° C., 25° C. to 37° C., 25° C. to 65° C., 25° C. to 90° C., 25° C. to 100° C., 37° C. to 65° C., 37° C. to 90° C., 37° C. to 100° C., 65° C. to 90° C., or 65° C. to 100° C.

### Diagnostic Apparatus

In some embodiments, a diagnostic system comprises one or more diagnostic apparatuses (e.g., a diagnostic apparatus comprising a lateral flow assay strip described herein). The one or more diagnostic apparatuses may comprise a “chimney” type diagnostic apparatus, a cartridge diagnostic apparatus, a blister pack diagnostic apparatus, and/or an integrated swab diagnostic apparatus.

In some embodiments, the diagnostic apparatus is a “chimney-type” diagnostic apparatus comprising a lateral flow assay strip. In certain embodiments, the “chimney-type” diagnostic apparatus comprises a chimney configured to receive a reaction tube. In certain embodiments, the “chimney-type” diagnostic apparatus further comprises a puncturing component (e.g., a puncturing protrusion) configured to puncture the reaction tube. The puncturing component may comprise one or more blades, needles, and/or one or more other elements or devices capable of puncturing material forming the reaction tube.

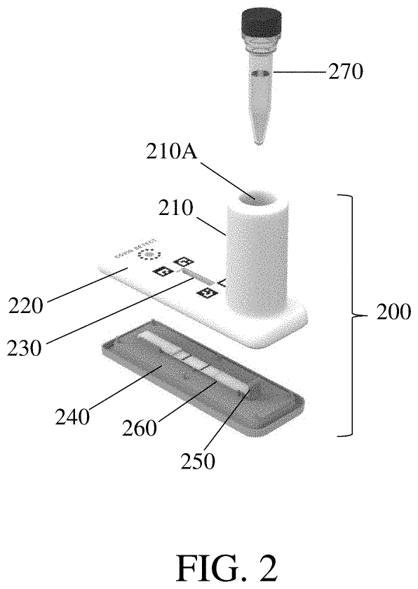

An exemplary “chimney-type” diagnostic apparatus 200 is shown in FIG. 2. In FIG. 2, “chimney-type” diagnostic apparatus 200 comprises chimney 210, front panel 220 comprising opening 230, and back panel 240 comprising puncturing protrusion 250 and lateral flow assay strip 260. In some embodiments, chimney 210 comprises an opening 210A configured to fit a reaction tube 270.

In operation, reaction tube 270 may contain fluidic contents (e.g., a fluidic sample) and may be inserted into opening 210A of chimney 210. Upon insertion, reaction tube 270 may be punctured by puncturing protrusion 250. As a result of puncturing, at least a portion of the fluidic contents may flow out of reaction tube 270 and may be deposited on a first sub-region (e.g., a sample pad) of lateral flow assay strip 260. In some embodiments, at least a portion of the fluidic contents of reaction tube 270 may flow or be transported through lateral flow assay strip 260 (e.g., via capillary action). In some embodiments, at least a portion of the fluidic contents of reaction tube 270 may flow through a second sub-region (e.g., a particle conjugate pad) of lateral flow assay strip 260. The second sub-region may comprise a plurality of labeled particles. In some embodiments, the fluidic contents of reaction tube 270 may comprise one or more amplified nucleic acids (e.g., amplicons). Flow of at least a portion of the fluidic contents through the second sub-region (e.g., particle conjugate pad) of lateral flow assay strip 260 may result in one or more amplicon-particle conjugates. In some embodiments, at least a portion of the amplicon-particle conjugates may flow through a third sub-region (e.g., a test pad) comprising one or more test lines that each may comprise one or more capture reagents (e.g., immobilized antibodies) configured to detect one or more target nucleic acid sequences, one or more nucleic acid control lines configured to detect one or more control nucleic acid sequences, and/or one or more flow control lines comprising one or more non-biological fluid indicators. In some embodiments, formation of one or more opaque lines at the one or more test lines may indicate a presence of one or more target nucleic acid sequences. Alternatively, in some embodiments, lack of formation of one or more opaque lines at the one or more test lines may indicate an absence of one or more target nucleic acid sequences. In some cases, formation of one or more opaque lines at the one or more nucleic acid control lines may indicate that a sample from the target species was successfully collected, nucleic acids from the sample were amplified, and the target amplification products were transported through the lateral flow assay strip. In some embodiments, formation of one or more opaque lines at the one or more flow control lines may indicate that a liquid (i.e., the fluidic sample) was successfully transported through the lateral flow assay strip. Alternatively, in some embodiments, lack of one or more opaque lines at the one or more nucleic acid control lines and/or flow control lines may indicate that the test results are invalid and may need to be re-run. In some embodiments, the one or more opaque lines, if present, may be visible through opening 230 of front panel 220.

In some embodiments, the diagnostic apparatus is a cartridge diagnostic apparatus comprising a lateral flow assay strip. The cartridge diagnostic apparatus may comprise a cartridge comprising one or more reagent reservoirs connected via one or more fluidic channels. The one or more reagent reservoirs may comprise one or more reagents (e.g., lysis reagents, nucleic acid amplification reagents) and/or one or more buffers. In certain embodiments, the cartridge may comprise a first reagent reservoir comprising a first set of reagents (e.g., lysis reagents) and/or a second reagent reservoir comprising a second set of reagents (e.g., nucleic acid amplification reagents). In some cases, the cartridge may further comprise one or more additional reagent reservoirs comprising one or more additional sets of reagents and/or buffers (e.g., a dilution buffer). The cartridge may, in some embodiments, also comprise one or more gas expansion reservoirs and/or vent paths configured to maintain a desired pressure in at least one reagent reservoir, one or more pumping tools (e.g., a peristaltic pump, a reciprocating pump) configured to facilitate fluid flow to and/or from one or more reagent reservoirs, and/or an integrated heater.

In some embodiments, the diagnostic apparatus is a blister pack diagnostic apparatus comprising a lateral flow assay strip. The blister pack diagnostic apparatus may comprise a blister pack comprising one or more chambers comprising one or more reagents (e.g., lysis reagents, nucleic acid amplification reagents) and/or one or more buffers (e.g., a dilution buffer). In certain embodiments, a chamber of the blister pack may be separated from an adjacent chamber by a breakable seal (e.g., a frangible seal) and/or a valve (e.g., a rotary valve).

In some embodiments, the diagnostic apparatus is an integrated swab diagnostic apparatus comprising a lateral flow assay strip. The integrated swab diagnostic apparatus may comprise a sample collection swab and a lateral flow assay strip comprising lysis and amplification stages integrated into a single device.

### Exemplary Diagnostic System

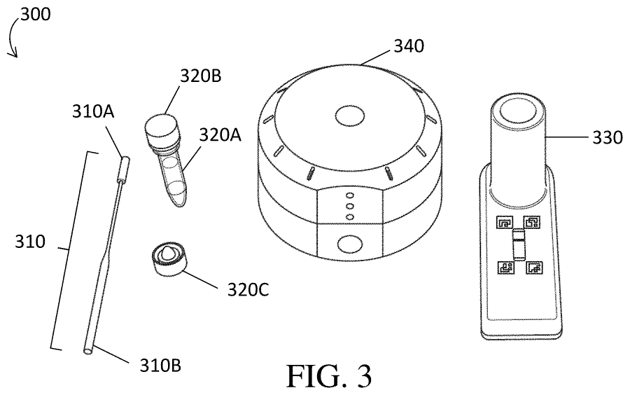

A non-limiting, illustrative embodiment of an exemplary diagnostic system utilizing one or more lateral flow assay strips is shown in FIG. 3. In FIG. 3, diagnostic system 300 comprises sample-collecting component 310, reaction tube 320, readout device 330, and heater 340. As shown in FIG. 3, sample-collecting component 310 may comprise swab element 310A and stem element 310B. Reaction tube 320 may comprise tube 320A, first cap 320B, and second cap 320C. First cap 320B and second cap 320C may independently be a screw-top cap or any other type of removable cap, and first cap 320B and second cap 320C may each be configured to fit over an opening of tube 320A. In some cases, first cap 320B and/or second cap 320C are airtight caps (e.g., configured to fit on tube 320A without any gaps). In some embodiments, second cap 320C comprises one or more reagents (e.g., lysis reagents, nucleic acid amplification reagents). The one or more reagents in cap 320C may be in solid form (e.g., lyophilized, dried, crystallized, air jetted) or in liquid form (e.g., in solution). In some cases, one or more reagents are solid and in the form of one or more pellets, capsules, gelcaps, and/or tablets. In certain instances, the one or more pellets, capsules, gelcaps, and/or tablets comprise one or more coatings (e.g., a coating of a time release material). In some embodiments, tube 320A comprises fluidic contents. In certain cases, the fluidic contents of tube 320A comprise one or more buffers (e.g., phosphate-buffered saline (PBS), Tris). In certain cases, the fluidic contents of tube 320A further comprise one or more salts (e.g., magnesium sulfate, ammonium sulfate, potassium chloride, potassium acetate, magnesium acetate tetrahydrate). In certain cases, the fluidic contents of tube 320A further comprise one or more detergents (e.g., Tween 20). The fluidic contents of tube 320A may have any suitable volume.

In operation, a user may collect a sample from a subject (e.g., a human subject, an animal subject) using sample-collecting component 310. In some cases, the subject is the user. In some cases, the subject is another human. In some instances, the user may insert swab element 310A into a nasal or oral cavity of the subject to collect a sample (and, in some cases, may self-collect a sample). After collecting a sample with swab element 310A, first cap 320B may be removed from tube 320A, and swab element 310A may be inserted into the fluidic contents of tube 320A. In some cases, the user may stir swab element 310A in the fluidic contents of tube 320A for a period of time (e.g., at least 10 seconds, at least 15 seconds, at least 20 seconds, at least 30 seconds). In some instances, swab element 310A is removed from tube 320A. In other instances, stem element 310B is broken and removed such that swab element 310A remains in reaction tube 320.

After swab element 310A and/or stem element 310B are removed from tube 320A, a cap may be placed on tube 320A. In some instances, for example, second cap 320C may be placed on tube 320A. In some cases, tube 320A and/or second cap 320C comprise one or more reagents (e.g., lysis reagents, nucleic acid amplification reagents). In some embodiments, one or more reagents may be released from second cap 320C into tube 320A by any suitable mechanism. In some cases, for example, the one or more reagents may be released into tube 320A by securing second cap 320C on tube 320A and inverting (and, in some cases, repeatedly inverting) reaction tube 320. In some cases, second cap 320C comprises a seal (e.g. a foil seal) separating the one or more reagents from the contents of tube 320A, and the seal may be punctured by screwing second cap 320C onto tube 320A, by puncturing the seal with a puncturing tool, or otherwise puncturing the seal. In some cases, the user presses on a button or other portion of second cap 320C and/or twists at least a portion of second cap 320C to release the one or more reagents into tube 320A.

In some embodiments, reaction tube 320 may be inserted into heater 340. Heater 340 may heat reaction tube 320 at one or more temperatures (e.g., at least 37° C., at least 63.5° C., at least 65° C.) for one or more periods of time. In some cases, heating reaction tube 320 according to a first heating protocol (e.g., a first set of temperature(s) and time period(s)) may reduce carryover contamination and/or facilitate lysis of cells within the collected sample. In a particular, non-limiting embodiment, a first heating protocol comprises heating reaction tube 320 at 37° C. for 3-10 minutes (e.g., about 3 minutes). In some cases, heating reaction tube 320 according to a second heating protocol (e.g., a second set of temperature(s) and time period(s)) may facilitate cell lysis and/or amplification of one or more target nucleic acids if present within the sample. In a particular, non-limiting embodiment, a second heating protocol comprises heating reaction tube 320 at 63.5° C. for 5-60 minutes (e.g., about 40 minutes). In some cases, heater 340 may comprise an indicator (e.g., a visual or audio indicator) that a heating protocol is occurring and/or has completed. The indicator may indicate to a user when reaction tube 320 should be removed from heater 340.

Following heating, reaction tube 320 may be inserted into readout device 330. Upon insertion, reaction tube 320 may be punctured by a puncturing component (e.g., a blade, a needle) of readout device 330. In some cases, puncturing reaction tube 320 may cause at least a portion of the fluidic contents of reaction tube 320 to be directed to flow towards (and come into contact with) a lateral flow assay strip of readout device 300. The fluidic contents of reaction tube 320 may flow through the lateral flow assay strip (e.g., via capillary action), and the presence or absence of one or more target nucleic acid sequences and/or control nucleic acid sequences may be indicated on a portion of the lateral flow assay strip (e.g., by the formation of one or more visual indicators on the lateral flow assay strip). In some cases, a flow control line comprising one or more non-biological fluid indicators may indicate whether the fluidic contents of reaction tube 320 were successfully transported through the lateral flow assay strip. In some instances, at least a portion of the lateral flow assay strip may be visible to a user through an opening of readout device 330. In some cases, software (e.g., a mobile application) may be used to read, analyze, and/or report the results (e.g., the one or more visual indicators of the lateral flow assay strip). In some embodiments, readout device 330 comprises one or more markings (e.g., ArUco markers) to facilitate alignment of an electronic device (e.g., a smartphone, a tablet) with readout device 330.

In some embodiments, a diagnostic system comprises a reaction tube comprising at least two caps that each comprise one or more reagents (e.g., lysis reagents, nucleic acid amplification reagents). In certain embodiments, the one or more reagents are in solid form (e.g., lyophilized, dried, crystallized, air jetted). In such embodiments, the at least two caps may be used to sequentially add reagents to a reaction tube.

### Instructions & Software

In some embodiments, a diagnostic system described herein may include instructions for using the components of the diagnostic system and/or otherwise performing a diagnostic test method. The instructions may include instructions for the use, assembly, and/or storage of the components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications). In some embodiments, the instructions are provided as part of a software-based application. In certain cases, the application can be downloaded to a smartphone or device, and then guides a user through steps to use the diagnostic device.

In some embodiments, a diagnostic system comprises or is associated with software to read and/or analyze test results. In some embodiments, a device (e.g., a camera, a smartphone) may be used to generate an image of a test result (e.g., one or more lines detectable on a lateral flow assay strip). In some embodiments, a user may use an electronic device (e.g., a smartphone, a tablet, a camera) to acquire an image of the visible portion of the lateral flow assay strip. In some embodiments, software running on the electronic device may be used to analyze the image (e.g., by comparing any lines or other markings that appear on the lateral flow assay strip with known patterns of markings). A machine vision software application may be employed to read the uploaded or entered test reading, and automatically provide a positive or negative test result. The result may be communicated directly to a user or to a medical professional. In some cases, the test result may be further communicated to a remote database server. In some embodiments, the remote database server stores test results as well as user information such as at least one of name, social security number, date of birth, address, phone number, email address, medical history, and medications. In other embodiments, the mobile application sends this information (e.g., an image of the resultant lateral flow test strip) to a secure, HIPAA-compliant, cloud-based software infrastructure. This software infrastructure then facilitates simple, fast, and scalable reporting to the federal and state health agencies.

In some embodiments, the database may generate a code based on the user's results (e.g., positive or negative for the viral illness). After a successful test, the code is available in the application. In some embodiments, the code may be read by a bar code scanner or other security detection device. If the user is negative for the viral illness and has a negative code, the security system will recognize the code and permit entry. In other embodiments, if the user is positive for the viral illness and has a positive code, the security system will recognize the code and deny entry.

### Methods of Making Diagnostic System Components

Certain aspects are directed to methods of making one or more components of a diagnostic system. In some embodiments, a method of making a diagnostic system comprises a method of preparing a lateral flow assay strip. In some embodiments, the method of preparing a lateral flow assay strip comprises contacting a substrate having a first end and a second end with one or more capture reagents (e.g., immobilized antibodies) to form a first test line at a first location on the substrate. In certain embodiments, the first test line is configured to detect a first target nucleic acid sequence. In some embodiments, the method of preparing a lateral flow assay strip comprises contacting the substrate with a non-biological fluid indicator to form a flow control line at a second location on the substrate. In certain embodiments, the second location is positioned between the first test line and the second end of the substrate.

In some embodiments, a method of making a diagnostic system comprises making a diagnostic apparatus. In some embodiments, the diagnostic apparatus comprises an upper component comprising a chimney and an opening (e.g., to allow visualizing of an underlying lateral flow assay strip) and a lower component comprising a lateral flow assay strip and one or more puncturing components. In some embodiments, a method of making a diagnostic apparatus comprises affixing a lateral flow assay strip described herein and/or a puncturing component to a lower component. In some embodiments, the method of making the diagnostic apparatus further comprises attaching an upper component to a lower component via one or more adhesives, one or more screws or other fasteners, and/or one or more interlocking components. In some embodiments, a method of making a diagnostic system further comprises providing any diagnostic system component described herein (e.g., a sample-collecting component, a reaction tube, a dropper, a pipette).

### Example 1

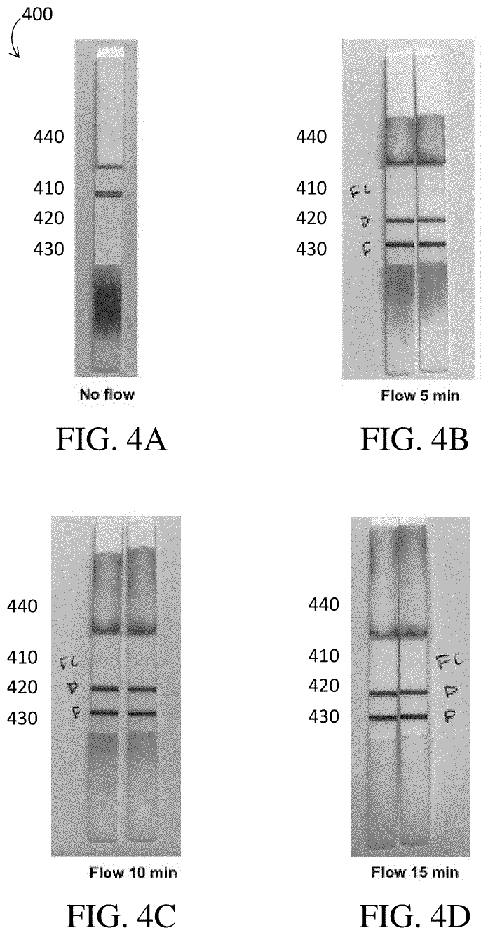

In this Example, a lateral flow assay (“LFA”) strip 400 was prepared with a flow control line 410 comprising FD&C Blue No. 1 (Brilliant Blue FCF, E133), a nucleic acid control line 420 comprising an anti-DIG antibody, a test line 430 comprising an anti-FITC antibody, and a wicking pad 440. FIG. 4A shows LFA strip 400 prior to application of a fluidic sample comprising SARS-CoV-2 amplicons labeled with FITC and RNaseP amplicons labeled with DIG. As shown in FIG. 4A, flow control line 410 was initially visible, and nucleic acid control line 420 and test line 430 were initially not visible.

The fluidic sample was then introduced to LFA strip 400. FIGS. 4B-4D show LFA strip 400 after 5 minutes of sample flow (FIG. 4B), 10 minutes of sample flow (FIG. 4C), and 15 minutes of sample flow (FIG. 4D). As shown in FIGS. 4B-4D, as the sample flowed through LFA strip 400, nucleic acid control line 420 and test line 430 became visible, and flow control line 410 became non-visible in its initial location. Instead, the food colorant of flow control line 410 became visible in wicking area 440 in FIGS. 4B-4D.

Thus, this Example demonstrates that a non-biological flow control line can demonstrate (e.g., through a visible change) whether a fluidic sample was successfully transported through a lateral flow assay strip.

It should be understood that the features and details described above may be used, separately or together in any combination, in any of the embodiments discussed herein.

Some aspects of the present technology may be embodied as one or more methods. Acts performed as part of a method may be ordered in any suitable way. Accordingly, embodiments may be constructed in which acts may be performed in an order different than described or illustrated, which may include performing some acts simultaneously, even though they may be shown or described as sequential acts in illustrative embodiments.

Aspects described in one embodiment may be combined in any manner with aspects described in other embodiments.

Any use of ordinal terms such as “first,” “second,” “third,” etc., in the description and the claims to modify an element does not by itself connote any priority, precedence, or order of one element over another, or the temporal order in which acts of a method are performed, but is or are used merely as labels to distinguish one element or act having a certain name from another element or act having a same name (but for use of the ordinal term) to distinguish the elements or acts.

The indefinite articles “a” and “an,” as used herein in the specification and in the claims, unless clearly indicated to the contrary, should be understood to mean “at least one.”

Any use herein, in the specification and in the claims, of the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Any use herein, in the specification and in the claims, of the phrase “equal” or “the same” in reference to two values (e.g., distances, widths, etc.) should be understood to mean that two values are the same within manufacturing tolerances. Thus, two values being equal, or the same, may mean that the two values are different from one another by ±5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. As used herein in the specification and in the claims, the term “or” should be understood to have the same meaning as “and/or” as defined above.

The terms “approximately” and “about” if used herein may be construed to mean within ±20% of a target value in some embodiments, within ±10% of a target value in some embodiments, within ±5% of a target value in some embodiments, and within ±2% of a target value in some embodiments. The terms “approximately” and “about” may equal the target value.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

What is claimed is:

1. A lateral flow assay strip, comprising:
a substrate having a first end and a second end, comprising:
a first test line configured to detect a first target nucleic acid sequence; and
a flow control line comprising a first non-biological fluid indicator,
wherein the flow control line is positioned between the first test line and the second end of the substrate,
wherein the substrate is configured to transport a fluidic sample from the first end to the second end.

2. The lateral flow assay strip of claim 1, wherein the first non-biological fluid indicator is configured to exhibit a visible change upon contact with the fluidic sample.

3. The lateral flow assay strip of claim 2, wherein the visible change comprises a change from a first color to a second color.

4. The lateral flow assay strip of claim 1, wherein the first non-biological fluid indicator comprises a pH-sensitive material.

5. The lateral flow assay strip of claim 4, wherein the pH-sensitive material comprises litmus, bromocresol purple, bromothymol blue, phenol red, and/or phenolphthalein.

6. The lateral flow assay strip of claim 1, wherein the first non-biological fluid indicator comprises a moisture-sensitive material.

7. The lateral flow assay strip of claim 6, wherein the moisture-sensitive material comprises CuCl₂ and/or CoCl₂.

8. The lateral flow assay strip of claim 1, wherein the first non-biological fluid indicator comprises a chemically-sensitive material.

9. The lateral flow assay strip of claim 8, wherein the chemically-sensitive material is configured to exhibit a visible change upon interaction with one or more salts, nucleotides, nucleic acids, and/or proteins.

10. The lateral flow assay strip of claim 1, wherein the first non-biological fluid indicator comprises a dye.

11. The lateral flow assay strip of claim 1, wherein the first target nucleic acid sequence is a nucleic acid sequence of a coronavirus and/or an influenza virus.

12. The lateral flow assay strip of claim 11, wherein the first target nucleic acid sequence is a nucleic acid sequence of SARS-CoV-2 and/or a SARS-CoV-2 variant.

13. The lateral flow assay strip of claim 1, further comprising a nucleic acid control line.

14. The lateral flow assay strip of claim 13, wherein the nucleic acid control line is configured to detect a target nucleic acid sequence of human RNase P.

15. The lateral flow assay strip of claim 1, further comprising a second test line configured to detect a second target nucleic acid sequence.

16. The lateral flow assay strip of claim 15, wherein the second target nucleic sequence is a nucleic acid sequence of a coronavirus and/or an influenza virus.

17. A diagnostic system, comprising:
one or more nucleic acid amplification reagents, wherein the one or more nucleic acid amplification reagents comprise a first primer directed to the first target nucleic acid sequence; and
a lateral flow assay strip according to claim 1.

18. The diagnostic system of claim 17, wherein the one or more nucleic acid amplification reagents comprise one or more isothermal nucleic acid simplification reagents.

19. A diagnostic method, comprising:
performing an isothermal nucleic acid amplification reaction configured to amplify a first target nucleic acid sequence; and
exposing a fluidic product of the isothermal nucleic acid amplification reaction to a lateral flow assay strip having a first end and a second end, wherein the lateral flow assay strip comprises:
a first test line configured to detect the first target nucleic acid sequence; and
a flow control line comprising a first non-biological fluid indicator, wherein the flow control line is positioned between the first test line and the second end of the substrate.

20. A method of preparing a lateral flow assay strip, comprising:
contacting a substrate having a first end and a second end with one or more capture reagents to form a first test line at a first location on the substrate, wherein the first test line is configured to detect a first target nucleic acid sequence; and
contacting the substrate with a non-biological fluid indicator to form a flow control line at a second location on the substrate, wherein the second location is positioned between the first test line and the second end of the substrate.
